(19) United States
(12) Patent Application Publication
(10) Pub. No.: US 2008/0208089 A1
(43) Pub. Date: Aug. 28, 2008

(54) Posture Support Device

(75) Inventors: Tracy Newkirk, Norfolk (GB); Hoi Kwan (James) Cheung, Etobicoke (CA)

Correspondence Address: BERESKIN AND PARR, 40 KING STREET WEST, BOX 401, TORONTO, ON, M5H 3Y2, CA

(73) Assignee: INTEGRAL ORTHOPEDICS INC., Toronto, ON (CA)

(21) Appl. No.: 11/817,577

(22) PCT Filed: Mar. 6, 2006
(86) PCT No.: PCT/IB06/01257

§ 371 Date: Aug. 31, 2007

Related U.S. Application Data
(60) Provisional application No. 60/658,843, filed on Mar. 4, 2005.

(51) Int. Cl. A61F 5/00

(52) U.S. Cl. 602/19; 602/20; 602/32; 602/36

(57) ABSTRACT

A support device for a wearer is provided, including an anchor member, a first strap and a second strap. The anchor member is fittable around the torso of the wearer. Each strap has a first end and a second end and connects to the anchor member at the first and second ends. Each strap has a shoulder-engaging portion that is shaped to engage a shoulder joint of the wearer. The straps are each adapted to carry a tensile force and thereby exert a rearward force on the shoulder joint through the shoulder-engaging portion. The straps are configured to engage the shoulders of the wearer no further medially than the acromioclavicular joints. Each strap extends along a path that, in use, does not cross over the anterior axillary folds of the wearer.

### FIELD OF THE INVENTION

The present invention relates to posture support devices and more particularly to posture support devices that are wearable.

### BACKGROUND OF THE INVENTION

It is generally recognized that poor posture can lead to several painful conditions over time. To assist in preventing poor posture several manufacturers have developed posture support devices for wear by people, and which are intended to improve the posture of the wearers. All of these devices, however, can create other problems for the wearer, such as reduced blood flow and such as painful compression of certain regions of the body of the wearer.

There is therefore a need for an improved posture support device.

### SUMMARY OF THE INVENTION

In a first aspect, the invention is directed to a posture support device for a wearer, including an anchor member, a first strap and a second strap. The anchor member is fittable around the torso of the wearer. Each strap has a first end and a second end and connects to the anchor member at the first and second ends. Each strap has a shoulder-engaging portion that is shaped to engage a shoulder joint of the wearer. The straps are each adapted to carry a tensile force and thereby exert a rearward force on the shoulder joint through the shoulder-engaging portion. The straps are configured to engage the shoulders of the wearer no further medially than the acromioclavicular joints. Each strap extends along a path that, in use, does not cross over the anterior axillary folds of the wearer.

In a second aspect, the invention is directed to a posture support device for a wearer, wherein the posture support device is configured to urge the shoulders of the wearer rearward while avoiding exerting a downward force on the clavicles of the wearer, and while avoiding crossing over the anterior axillary folds of the wearer.

In a third aspect, a posture support device for a wearer, including an anchor member, a first strap and a second strap. The anchor member is fittable around the torso of a wearer. Each strap has a first end and a second end and connects to the anchor member at the first and second ends. Each strap has a shoulder-engaging portion that is shaped to engage a shoulder joint of the wearer. The straps are each adapted to carry a tensile force and thereby exert a rearward force on the shoulder joint through the shoulder-engaging portion. The straps are configured to engage the shoulders of the wearer no further medially than the acromioclavicular joints. The posture support device is free of any tension bearing members that encircle the thorax of the wearer either alone or in combination, thereby facilitating expansion of the rib cage during breathing.

In one embodiment of the third aspect, the anchor member has an upper edge which extends across the front of the torso of the wearer below the xiphoid process and no lower than the bottom rib and wherein the anchor member is adapted to carry a tensile force and thereby urge the ribs upwards in use to increase the volume of the thoracic cage.

In a fourth aspect, the invention is directed to a posture support device for a wearer, including an anchor member, a first strap and a second strap. The anchor member is fittable around the torso of the wearer. Each strap has a first end and a second end and connects to the anchor member at the first and second ends. Each strap has a shoulder-engaging portion that is shaped to engage a shoulder joint of the wearer. The straps are each adapted to carry a tensile force and thereby exert a rearward force on the shoulder joint through the shoulder-engaging portion. The straps are configured to engage the shoulders of the wearer no further medially than the acromioclavicular joints. The posture support device further comprises at least one covering panel. The at least one covering panel is connected to at least one of the anchor member, the first strap and the second strap while transferring substantially no force between the wearer and any of the anchor member, the first strap or the second strap. The at least one covering panel is configured to cover portions of the body of the wearer which the anchor member and the first and second straps are not configured to cover.

### BRIEF DESCRIPTION OF THE DRAWINGS

The present invention will now be described by way of example only with reference to the attached drawings, in which:

FIG. 1a is a perspective view of a person exhibiting good posture, in accordance with the prior art;

FIG. 1b is a rear elevation view of the person shown in FIG. 1a;

FIG. 1c is a magnified rear elevation view of a scapula, a clavicle, a humerus and a sternum from the person shown in FIG. 1a;

FIG. 1d is a magnified rear elevation view of some of the muscles, neurovascular structures and skeletal structure of the person shown in FIG. 1a;

FIG. 1e is a front elevation view of some of the internal musculature on the person shown in FIG. 1a;

FIG. 1f is a front elevation view of some of the skeletal structure of the person shown in FIG. 1a illustrating the passage of some neurovascular structures through a gap between the clavicle and the first rib;

FIG. 2a is a rear elevation view of person shown in FIG. 1a, showing abduction of the scapulae resulting from poor posture;

FIG. 2b is a front elevation view of the skeletal structure shown in FIG. 1f illustrating the compression of some neurovascular structures between the clavicle and the first rib;

FIG. 3a is a front elevation view of a support device in accordance with an embodiment of the present invention;

FIG. 3b is a front elevation view of the support device shown in FIG. 3a, being worn by a wearer;

FIG. 3c is a rear elevation view of the support device shown in FIG. 3a, being worn by the wearer;

FIG. 3d is a magnified front elevation view of a portion of the support device shown in FIG. 3a on the wearer, showing some of the skeletal structure of the wearer;

FIG. 3e is a front elevation view of the support device shown in FIG. 3a, being worn by a wearer, with an optional strap member;

FIG. 3f is a rear elevation view of the support device shown in FIG. 3a, being worn by the wearer; with another optional strap member;

FIG. 3g is a front elevation view of a the support device shown in FIG. 3a, relative to the skeletal structure of the wearer;

FIG. 4a is a front elevation view of a support device in accordance with another embodiment of the present invention, being worn by a wearer;

FIG. 4b is a rear elevation view of the support device shown in FIG. 4a;

FIG. 5 is a rear elevation view of an optional sleeve portion that can be included with the support devices shown in FIGS. 3a and 4a;

FIG. 6a is a front elevation view of a support device in accordance with another embodiment of the present invention, being worn by a wearer;

FIG. 6b is a rear perspective view of the support device shown in FIG. 6a, illustrating the tensioning member in a closed position;

FIG. 6c is a rear perspective view of the support device shown in FIG. 6a, illustrating a tensioning member in an open position;

FIG. 7 is a rear perspective view of an alternative tensioning member that could be used with the support device shown in FIG. 6a; and

FIG. 8 is a front perspective view of a support device on a female wearer, in accordance with another embodiment of the present invention.

### DETAILED DESCRIPTION OF THE INVENTION

For purposes of clarity the terms ‘circumference’ and ‘effective circumference’ are used herein to refer to the distance around the perimeter of an object or a portion of the human body, even though the object or the body portion may not be perfectly circular.

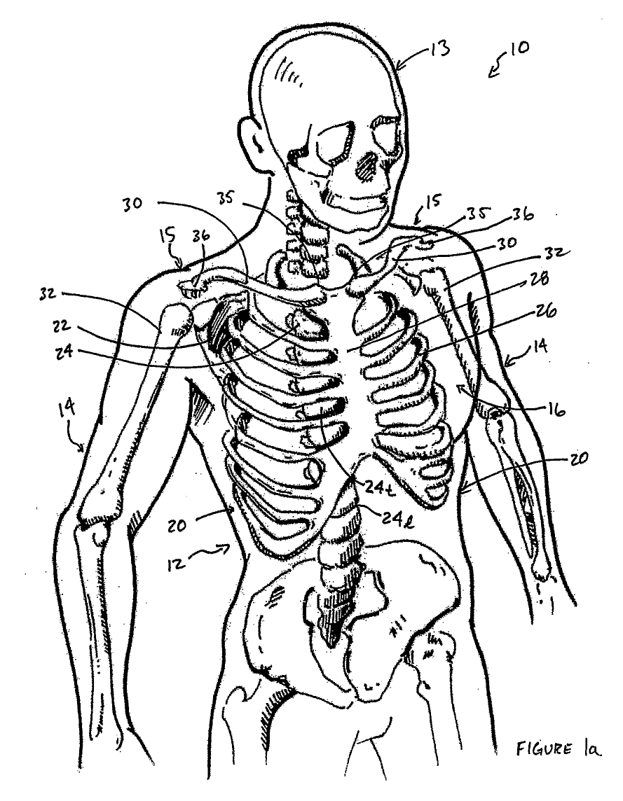

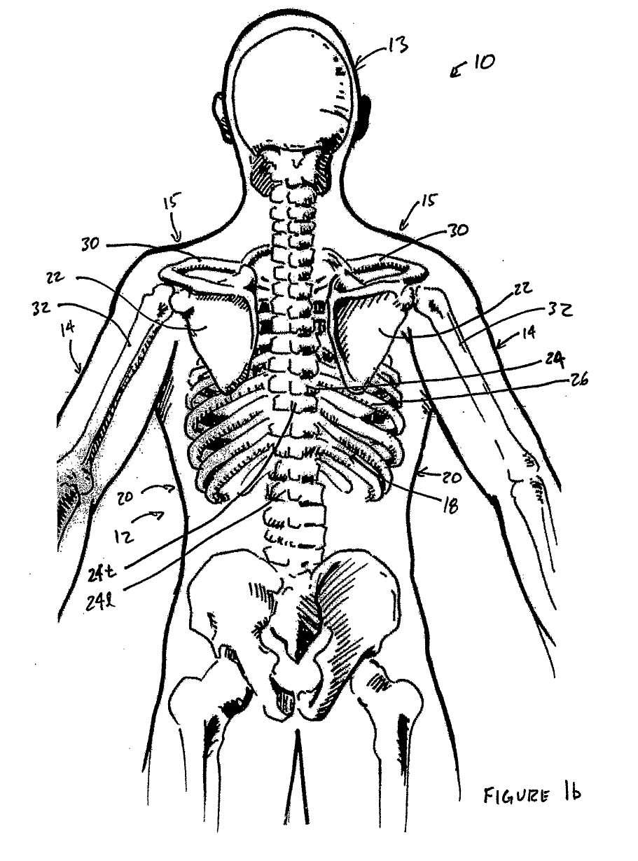

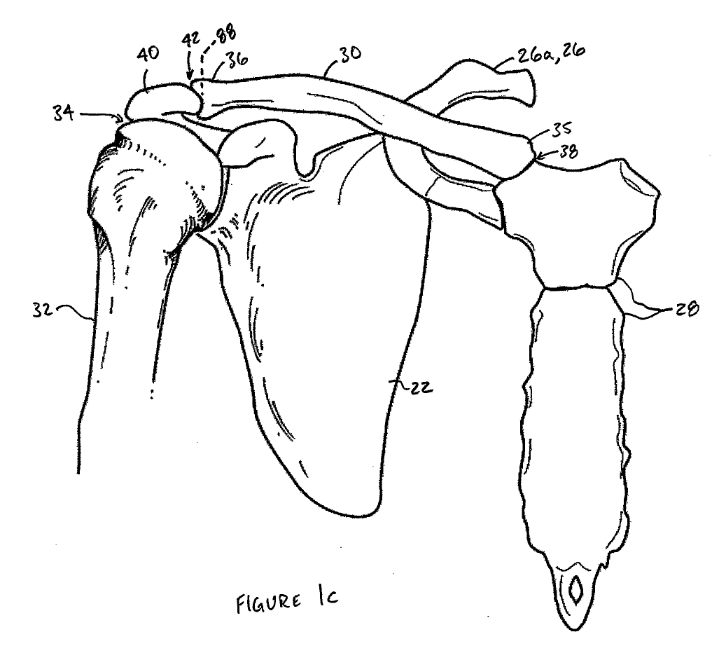

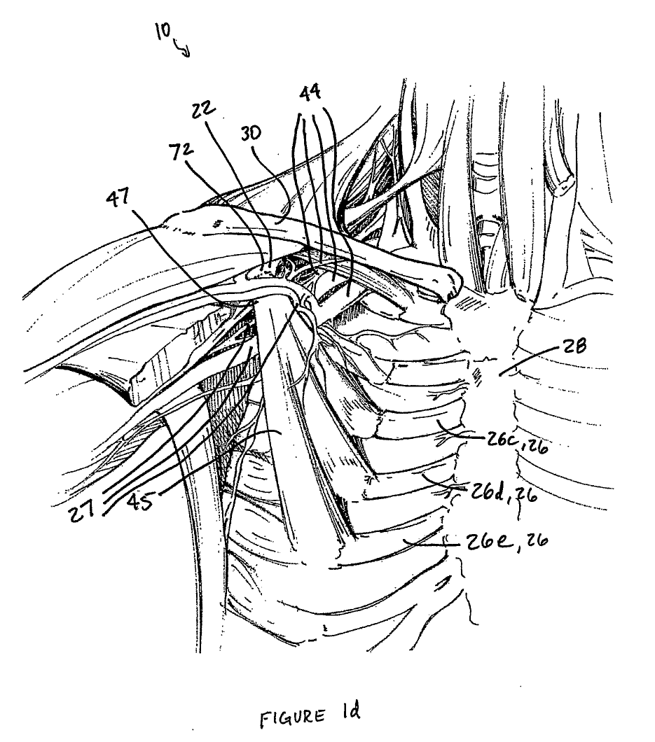

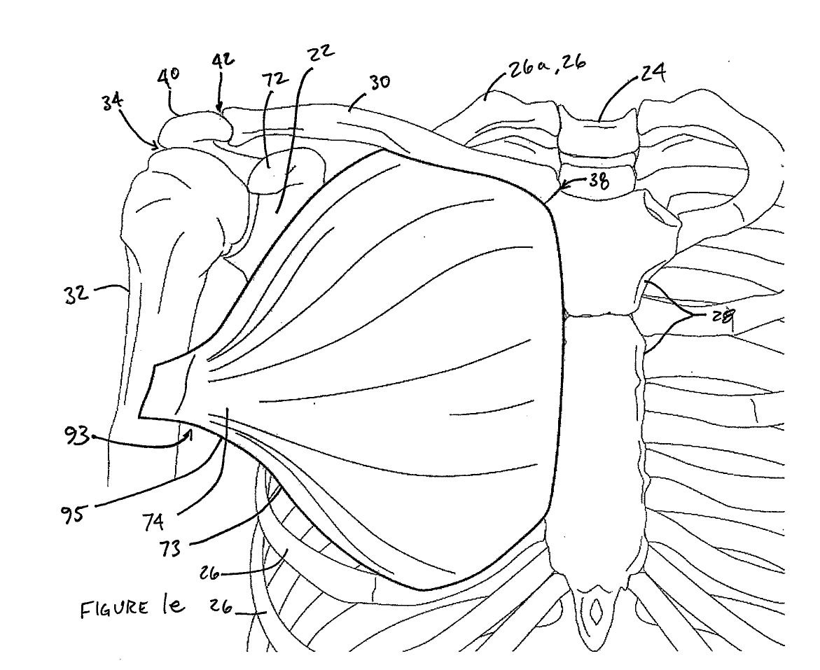

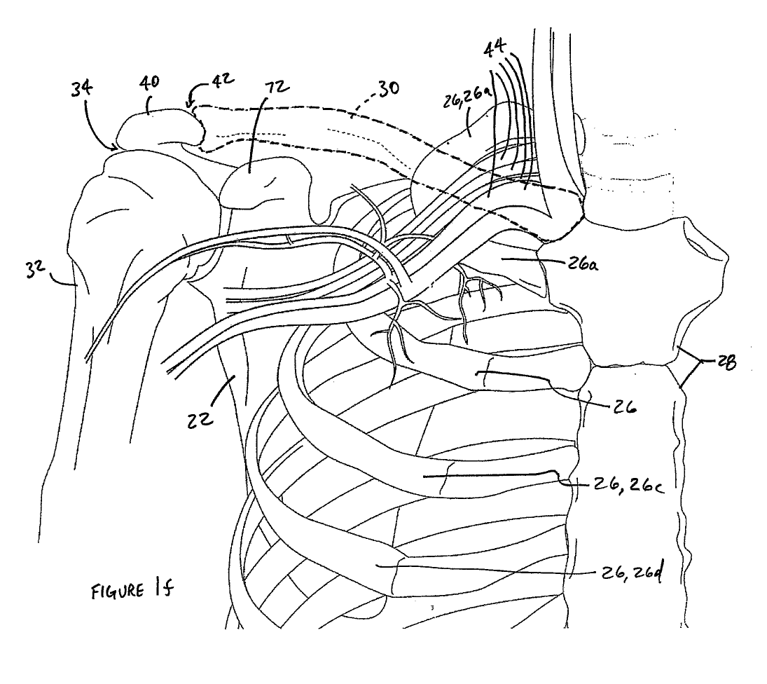

Reference is made to FIGS. 1a and 1b, which show a person 10. The person 10 has a body that includes, among other things, a torso 12, a head 13, a pair of arms 14 and a pair of shoulders 15. The torso 12 has a front 16 (FIG. 1a), a back 18 (FIG. 1b) and two sides 20. Skeletal elements that are present in the torso 12 and arms 14 include, among other things, two scapulae 22 (FIG. 1b), a spine 24, ribs 26, a sternum 28 (FIG. 1a), two clavicles 30 and two humeruses 32.

Referring to FIG. 1c, the scapula 22 and the humerus 32 meet at a joint 34, which is the shoulder joint 34, also known as the glenohumeral joint 34. Each clavicle 30 has a first end 35 and a second end 36. At the first end 35, the clavicle 30 is connected at a joint 38 to the top of the sternum 28. At the second end 36, the clavicle 30 is connected to a region of the scapula 22 called the acromion, shown at 40. The joint between the clavicle 30 and the acromion 40 is referred to as the acromioclavicular joint and is shown at 42. The medial edge of the acromioclavicular joint 42 is shown at 88.

The first rib is shown at 26a in FIG. 1c, and connects to the sternum 28 just under the clavicle 30. A gap is present between the clavicle 30 and the first rib 26a.

Referring to FIG. 1d, the pectoralis minor muscle, shown at 45, is connected at one end to a pectoralis minor tendon 47, which is itself connected to a portion of the scapula 22 known as the coracoid process, shown at 72. At its other end, the pectoralis minor 45 connects to the third, fourth and fifth ribs, which are shown at 26c, 26d and 26e.

Referring to FIG. 1e, the pectoralis major muscle is shown at 74 and extends between the sternum 28, the clavicle 30 and the humerus 32. The lateral border of the pectoralis major 74 is shown at 73. A portion 95 of the lateral border 74 of the pectoralis major 74 forms the anterior axillary fold, which is an edge of the axilla, shown at 93.

Referring to FIG. 1d, several neurovascular structures pass through the gap, passing over first rib 26a and under the clavicles 30. These neurovascular structures are identified at 44, and include the brachial plexus, the subclavian vein, and the subclavian artery. Additionally, the subclavius muscle is present in the gap, and thus occupies some of the space between the clavicle 30 and the first rib 26a.

Several neurovascular structures pass underneath the pectoralis minor tendon 47, including the brachial plexus and the axillo-subclavian artery and vein. These neurovascular structures are identified at 27.

Referring to FIGS. 1a and 1b, the torso 12 has a first position, in which the person 10 can be described as having good posture. When exhibiting good posture, the lumbar and thoracic portions of the spine 24 are together generally S-shaped, curving outwards anteriorly in the lumbar region shown at 24l and curving outwards posteriorly in the thoracic region, shown at 24t .

Referring to FIG. 1f, when the person 10 exhibits good posture, sufficient space is present in the gap between the clavicles 30 and the first rib 26a to permit the vascular structures 44 that pass through substantially uncompressed.

Additionally, referring to FIG. 1d, when the person 10 exhibits good posture, sufficient space exists under the pectoralis minor tendon 47 to permit the passage underneath of the neurovascular structures 27 with substantially no compression.

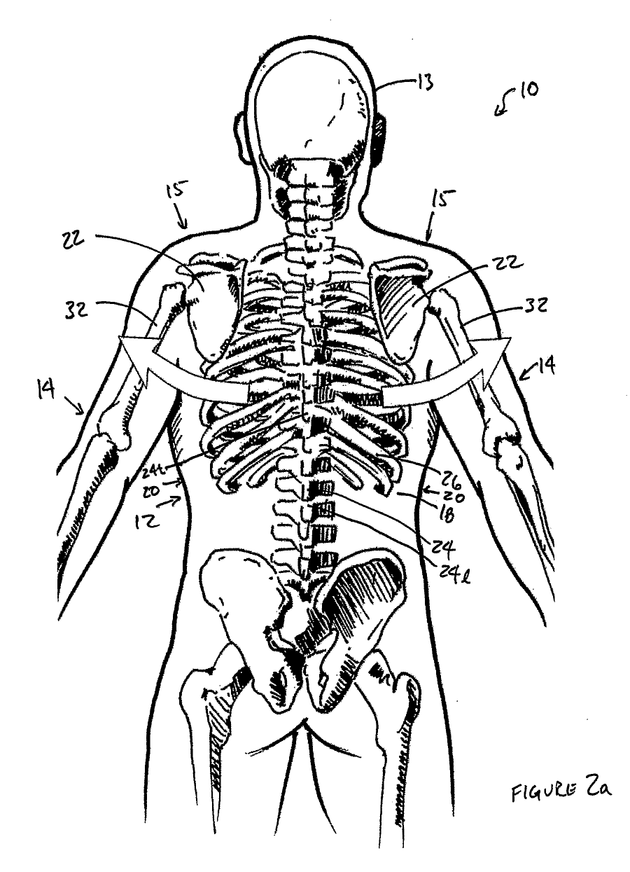

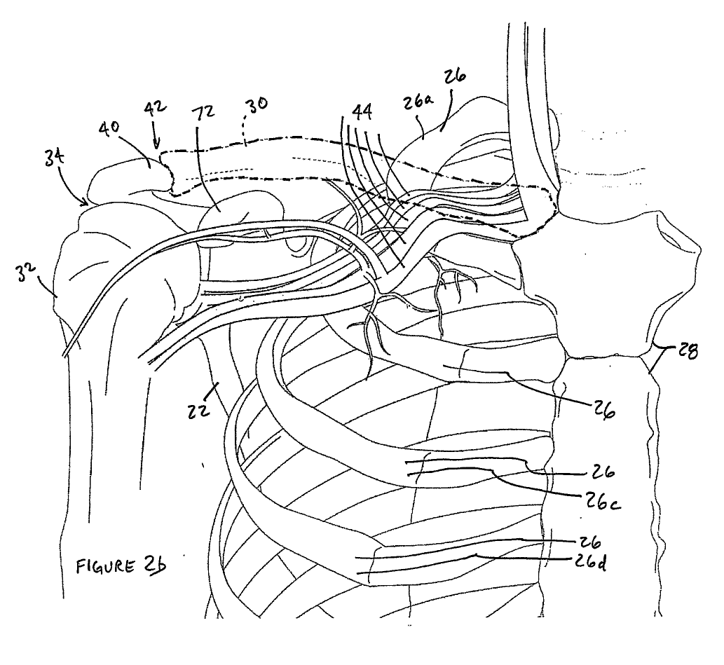

Referring to FIG. 2a, when a person 10 is in a slouched, or round-shouldered, position, numerous ailments can result, including, for example, migraine headaches, blurred vision, facial pressure, tinnitus, neck pain, neck swelling, hand and arm pain, coldness of the hands and/or feet, back pain, groin pain and intermittent numbness.

In a slouched, or round-shouldered, position, the body, and in particular, the spine 24 no longer has an S-shaped configuration. As a result, pressure is distributed between the vertebrae and the discs of the spine 24 in such a way as to urge the discs to bulge, which can, over time, lead to several problems, which are known to persons skilled in the art.

A round-shouldered posture may be acquired as a result of lifestyle and/or occupation. For example, in some types of employment, a person 10 uses their pectoral muscles 45 and 74 predominantly and the periscapular muscles and lumbar extensors are typically neglected, resulting in what is called deconditioning. In a deconditioned state the combined pull of the pectorals 45 and 74 and the failure of the periscapular muscles and lumbar extensors together create a round-shouldered posture.

As a result of the relatively poorly opposed pull of the pectorals 45 and 74, the scapula 22 becomes abducted, and thus moves outwards laterally, as shown in FIG. 2b. The lateral movement of the scapula 22 causes the clavicle 30 to pivot generally in a horizontal plane about the acromioclavicular joint 42, whereby its first, or proximal end 35 is urged rearwardly. This changes the angle of the clavicle 30 in the horizontal plane relative to the first rib 26a, and results in a narrowing of the gap. This, in turn, results in compression of some or all of the neurovascular structures 44 that pass through the gap, such as, for example, the brachial plexus and the subclavian vein. The subclavian artery may be compressed, but this may be less common due to several reasons, one of which is that the wall of the subclavian artery is relatively more rigid than at least some of the other neurovascular structures 44.

It will be understood that, when compression of the neurovascular structures 44 is said to take place between the clavicle 30 and the first rib 26a, one or both of the clavicle 30 and the first rib 26a might not be in direct contact with the structures 44 that are being compressed. Other elements such as the subclavian muscle or the infraspinatus which are present in the gap may be involved in the compression.

Compression of the neurovascular structures 44 can lead to several of the ailments noted above. For example, as a result of the compression of the vascular structures that are included in the neurovascular structures 44, the resistance to fluid flow in these vascular structures increases, which results in dilation of certain veins and tributaries, and increased arterial vascular resistance due at least in part to the slowed venous flow. The consequences of venous congestion and increased arterial resistance are well known in the form of edema and ischemia first in the areas of greatest vascular impairment and later in other body regions also. Numerous ailments can result from this condition of compressed neurovascular structures 44, including, for example, migraine headaches, facial pressure, neck pain, hand and arm pain and numbness, coldness of the hands, some forms of non-radicular leg pain and intermittent numbness.

As a result of its connection with the sternum 28, the rearward movement of the first or proximal end 35 of the clavicle 30 causes the sternum 28 (and in particular, the manubrium sternum) to move rearwardly along with it, thus reducing the distance between the manubrium sternum and the anterior border of the body of T3 (the third thoracic vertebra).

In certain types of people 10 this reduction in the aforementioned diameter can lead to one or more difficulties. For example, individuals with a thin, narrow thorax tend to have a relatively straight cervical and thoracic spinal, saggital alignment. In such people, a loss of cervical lordosis and flattening of the thoracic kyphosis leads to a closer approximation of the posterior surface of the manubrium sternum to the anterior border of the body of the third thoracic vertebrae. For example, in some people this distance may be less than approximately 5 cm (2 inches). With the proximal ends 35 of the clavicles 30 urged rearwardly as described above, this distance may be reduced to approximately 2.5 cm (1 inch) to 3.8 cm (1.5 inches). This is especially true at the head of the first rib 26a which sits deeper into the thorax than the other ribs 26. This positioning of the first rib 26a permits it to compress particular veins, such as the brachiocephalic vein. Other people 10 who may be prone to incurring difficulties from the reduced ‘diameter’ include individuals who have pectus excavatum, individuals who have thoracic scoliosis, individuals who have a loss of cervical lordosis in general, and/or individuals who are obese.

In addition to causing the rotation of the clavicle 30 in the horizontal plane, the movement of the scapula 30 as a result of deconditioning can also cause a rotation of the clavicle 30 generally about its own length in a clockwise direction from a viewpoint facing the side of the left shoulder of the person 10, and in a counterclockwise direction from a viewpoint facing the side of the right shoulder of the wearer 10. Due to the configuration of the clavicle 30, this rotation further narrows the gap between the clavicle 30 and the first rib 26a, thereby adding to the compression of the neurovascular structures 44 that pass therethrough.

In addition to the above, deconditioning of the stabilizer muscles of the scapula 22 can also lead to ‘non-neurogenic winging’ of the scapula 22. Non-neurogenic winging of the scapula 22 refers to an outward movement of the medial edge of the scapula 22 away from the plane of the back 18, however, it is not caused by damage to the long thoracic nerve, which is the typical cause of neurogenic winging. Non-neurogenic winging is instead the result of the weakened state of the stabilizer muscles that hold the medial edge of the scapula 22 down in the plane of the back 18.

Non-neurogenic winging of the scapula 22 causes a rotary torque to be exerted on the acromioclavicular joint 42, which in turn causes a downward movement of the clavicle 30. This downward movement of the clavicle 30 further reduces the size of the gap, and therefore exacerbates the problem of compression of the neurovascular structures 44 that pass therethrough.

Additionally, in situations where drooping of the scapula 22 occurs as a result of deconditioning, the coracoid process 43 and, in turn, the pectoralis minor tendon 47 may be lowered relative to their normal position, whereby the pectoralis minor tendon 47 compresses the neurovascular structures 27 that pass underneath. Compression of these structures 27 results in many of the same ailments as compression of the structures 44 that pass underneath the clavicles 30.

In addition to the above, individuals 10 who have a round-shouldered posture are typically relatively sensitive along the anterior axillary fold 95, such that, any compression of the anterior axillary fold 95 results in pain.

It will be noted that movement of the scapulae 22 can impart movement to the ribs 26 by way of their connection to the ribs 26 through the scapular stabilizer muscles. Abduction of the scapulae 22, as shown in FIG. 2a can urge the fronts of the ribs 26 to turn downwards at their joints with the sternum 28. This can result in a reduction in the volume of the rib cage, which can add resistance to expansion of the lungs during breathing, thereby hampering breathing

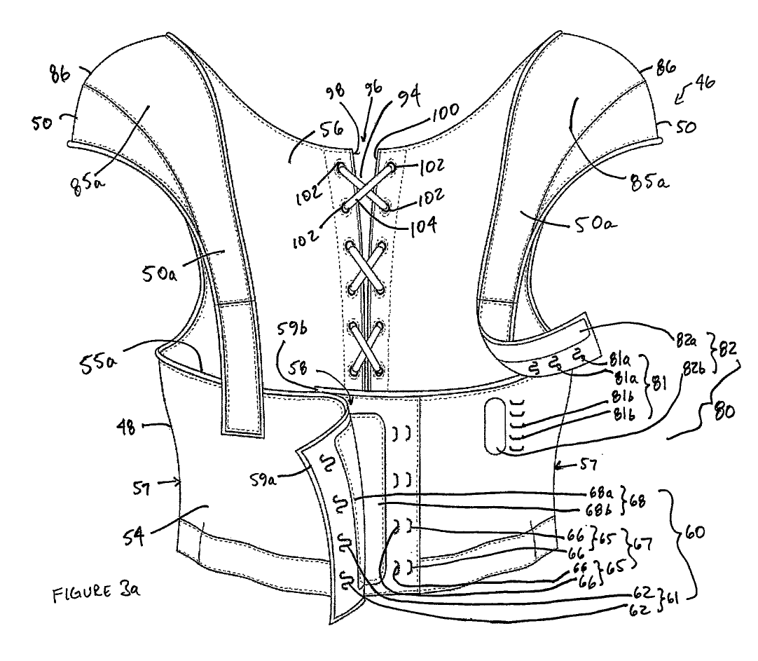

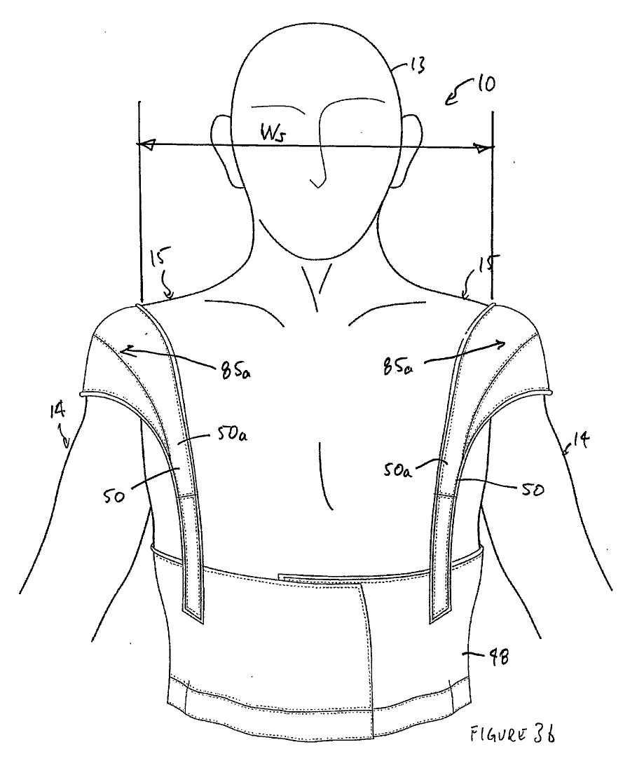

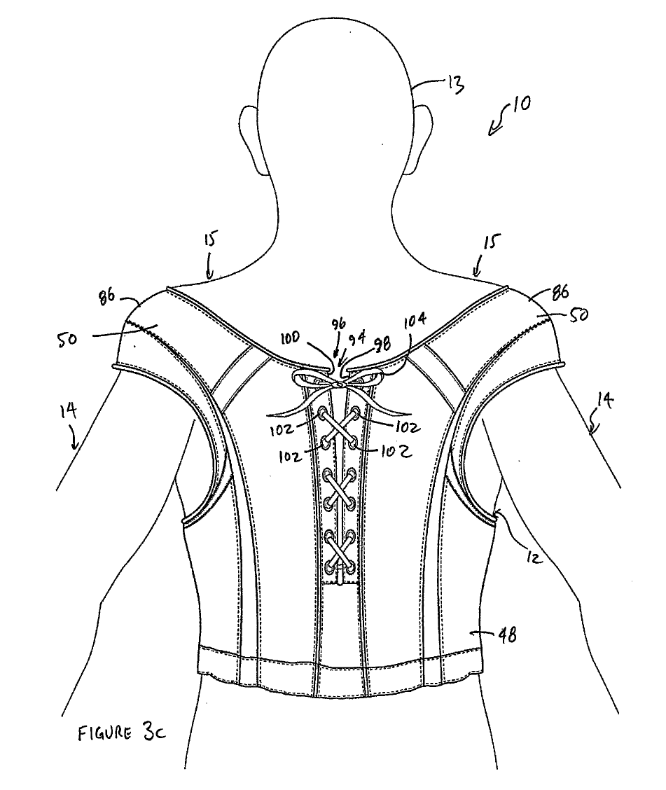

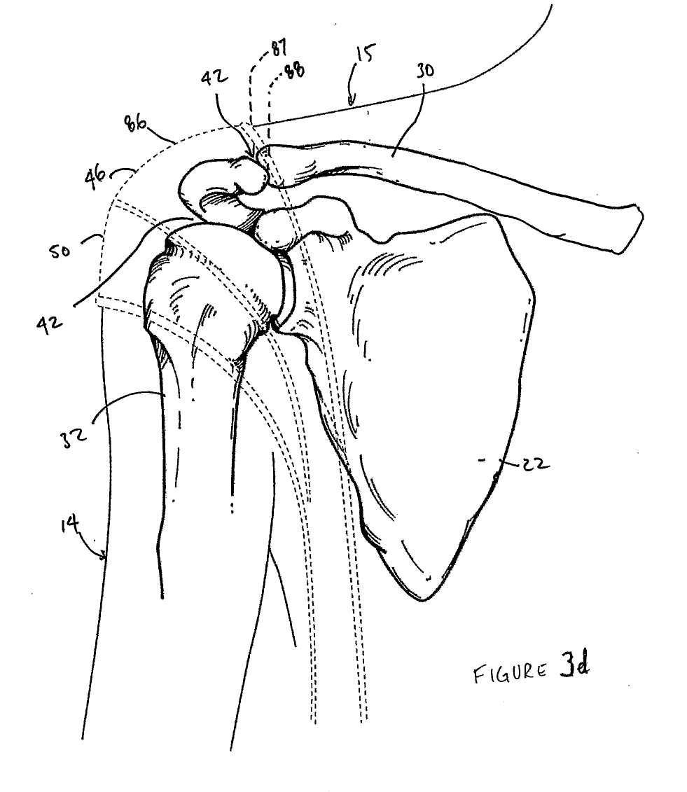

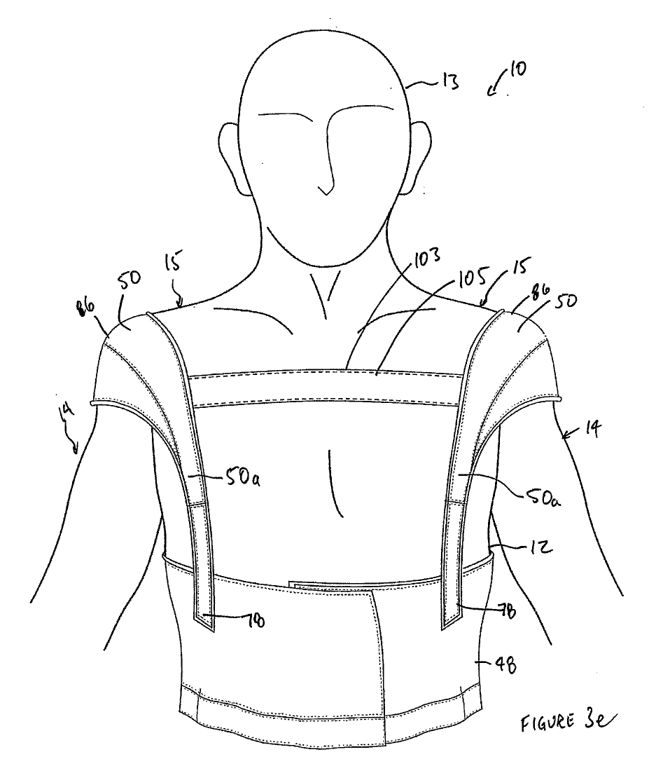

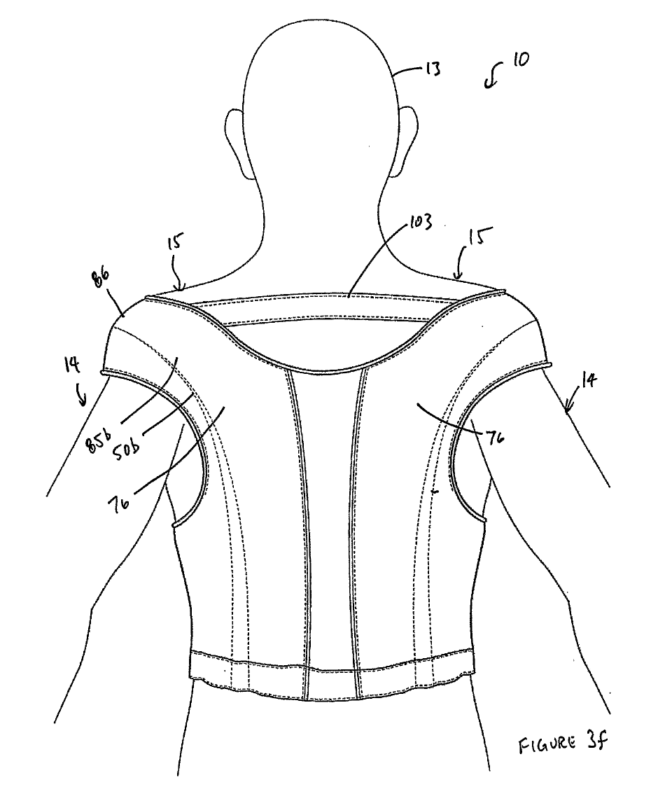

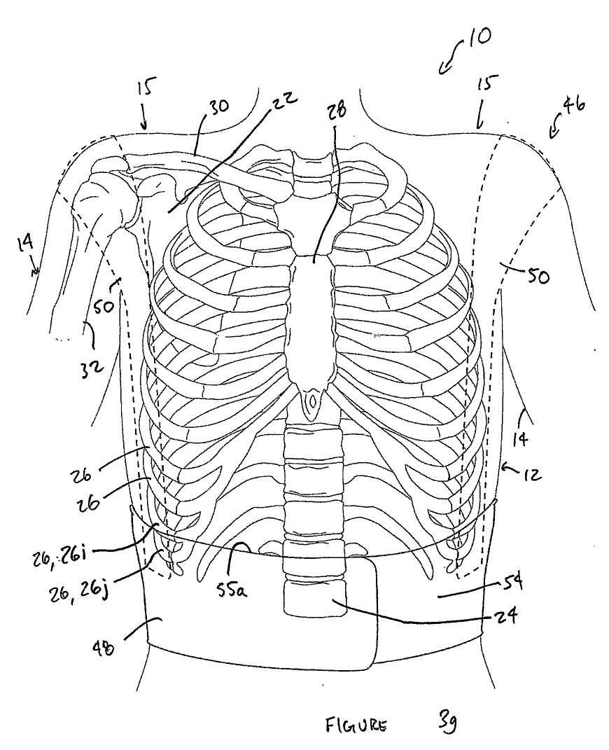

Reference is made to FIGS. 3a, 3b and 3c, which show a support device 46 in accordance with a first embodiment of the present invention. When worn, the support device 46 improves some or all of the ailments described above relating to poor posture.

The support device 46 includes an anchor member 48, and two straps 50. Referring to FIG. 3b, the anchor member 48 is fittable around the torso 12 of the wearer 10, and acts as an anchor for the straps 50. The anchor member 48 has a front 54, a back 56 and two sides 57.

The anchor member 48 may optionally include an opening 58, which is closable by a connector 60 (FIG. 3a). The opening 58 may be provided at any suitable position about the circumference of the anchor member 48. For example, the opening 58 may be provided at the front 54 of the anchor member 48 to facilitate access to the connector by the wearer 10 when wearing the support device 46. The connector 60 may be made up of any suitable structure. For example, the connector 60 may be made up of a set 61 of one or more hooks 62 proximate a first edge 59a of the anchor member 48, and a set 65 of one or more eyes 66 proximate a second edge 59b of the anchor member 48. The hooks 62 are engageable with the eyes 66 to secure the anchor member 48 around the torso 12 of the wearer 10.

To prevent the one or more hooks 62 from disengaging from the one or more eyes 66 inadvertently, the connector 60 may be supplemented by a hook-and-loop portion 68, which is positioned proximate the hooks 62 and eyes 66. The hook-and-loop connector portion 68 includes a hook portion 68a, which may be proximate the one or more hooks 62 and a loop portion 68b, which may be positioned proximate the one or more eyes 66.

Other connectors 60 are alternatively possible. For example, the connector 60 may be made solely of a strip of hook material 68a and a strip of loop material 68b, which may form a sufficiently strong connection without the use of hooks and eyes. As yet another example of a connector 60, the connector 60 may include a zipper instead of the aforementioned hooks and eyes and instead of the aforementioned hook material and loop material.

The anchor member 48 may further include a circumference adjustment device 67, which permits the effective circumference of the anchor member 48 to be adjusted to accommodate wearers 10 of different sizes. The circumference adjustment device 67 may be made up by any suitable structure. For example, the anchor member 48 may include a plurality of sets 65 of one or more eyes 66, wherein each set 65 is positioned at a different distance from the second edge 59b. For example, a first set 65 may be positioned approximately 1 cm from the second edge 59b, a second set 65 may be positioned approximately 3 cm from the second edge 59b. The set 61 of one or more hooks 62 could be insertable into any set 65 of one or more eyes 66, thereby adjusting the effective circumference of the anchor member 48.

It is alternatively possible to provide the one or more hooks 62 proximate the second edge 59b and to provide the one or more eyes 66 proximate the first edge 59a.

It is alternatively possible for the circumference adjustment device 67 to include a plurality of sets 61 of hooks 62, wherein the sets 61 are positioned at different distances from their respective end 59a or 59b of the anchor member 48, and to include a single set 65 of eyes 66 proximate the other end 59a or 59b of the anchor member 48. It is further alternatively possible for the circumference adjustment device 67 to include a plurality of sets 61 of hooks 62 and a plurality of sets 65 of eyes 66.

If strips of hook material 68a and loop material 68b are included in the connector 60, these strips 68a and 68b can be made to engage each other no matter which set 61 of hooks 62 and which set 65 of eyes 66 are engaged.

Referring to FIG. 3g, the upper edge of the anchor member 48 is shown at 55a. Preferably, at the front 54 of the anchor member 48, the top edge 55a engages the wearer 10 below the eighth rib, shown at 26h. For example, the upper edge 55a may be configured to engage the wearer 10 just above the bottom of the ninth rib, shown at 26i. As a result of its engagement with the ninth rib 26i, the anchor member 48 urges the ribs 26 upwards, which can increase the volume of the rib cage, which in turn can facilitate lung expansion during breathing.

Instead of engaging the ninth rib 26i, the top edge 55a of the anchor member 48 may be configured to engage the wearer 10 just above the bottom of the tenth rib, shown at 26j, and to urge the ribs 26 upwards by that engagement.

The anchor member 48 may be made from any suitable material, and is preferably made from a material with some elasticity to permit it to fit snugly around the wearer 10. A snug fit assists the anchor member 48 in engaging the torso 12 and in resisting riding up on the wearer 10 during use.

Referring to FIG. 3b, the straps 50 extend over the shoulders 15 and engage the shoulders joints 34. Each of the straps 50 has a first end 76 (FIG. 3c) and a second end 78 (FIG. 3b). The first and second ends 76 and 78 are connected to the anchor member 48. The first end 76 may be connected to the back 56 of the anchor member 48 (FIG. 3c). The second end 78 may be connected to the front 54 of the anchor member 48 (FIG. 3b).

The straps 50 each have a shoulder-engaging portion 86 which engages one of the shoulder joints 34 (see FIG. 3d) of the wearer 10. The shoulder-engaging portion 86 has a front end 85a (FIG. 3b) and a rear end 85b (FIG. 3c). Each strap 50 has a rear portion 50b (FIG. 3c) that extends from the rear end 85b of its shoulder-engaging portion 86 to the back 56 of the anchor member 48.

Referring to FIG. 3b, the support device 46 has a width Ws between the medial edges of the straps 50. This width Ws is selected so that the straps 50 each carry a selected tensile force and thereby urge the shoulder joints 34 rearwardly. Reduction in this selected width Ws increases the force with which the straps 50 urge the shoulder joints 34 rearward.

Referring to FIG. 1, each strap 50 has a front portion 50a that extends from the front end 85a of its shoulder-engaging portion 86 to the front 54 of the anchor member 48.

The angle of the rear portion 50b may be selected to be non-vertical. By selecting the angle to be non-vertical, some of the force exerted by the rear strap portion 50b on the shoulder joint 34 (through the shoulder engaging portions 86) is in the medial direction. Thus, the straps 50 are, in such an embodiment, adapted to carry a tensile force that urges the shoulder joints 34 medially.

To facilitate the carrying of tension in the straps 50, the straps 50 may be made from a material that has a selected amount of elasticity, and a selected ratio of elastic tension per unit of stretch. For example, a suitable material may be Supplex™, (ie. the same material as can be used for the anchor portion 48. Another suitable material for the device 48 may be, for example, lingerie-grade Spandex™.

The support device 46 may include a width control device 94 to permit the effective width of the support device 46 to be controlled. This permits control over the tension in the rear strap portions 50b.

Additionally, this permits the support device 46 to accommodate a plurality of wearers 10 having a range of shoulder widths. The width control device 94 may be positioned anywhere suitable, such as on the back 56 of the anchor member 48. The width control device 94 may have any suitable structure. For example, the anchor member 48 may have a separation 96 that extends at least partially down the back 56. The separation 96 may be defined on one side by a first edge 98 of the anchor member 48, and on the other side by a second edge 100 of he anchor member 48. A set of apertures 102 may be provided proximate each edge 98 and 100 of the anchor member 48. A tie member 104 passes through the apertures 102 and is tied to control the size of the separation 96, thereby controlling the distance Ws.

Preferably, the shape of the separation 96, formed by the edges 98 and 100 is a V-shape. A V-shape reduces the amount of bunching that might occur if the edges 98 and 100 were parallel and the separation 96 were generally rectangular.

The width control device 94 in combination with the straps 50 and the back 56 of the anchor member 48 act to adduct the scapulae 22. Additionally, by tightening the width control device 94, the straps 50 will urge the shoulders 15 back further and will further adduct the scapulae 22. Thus the urging force on the scapulae 22 and in turn the amount of adduction can be controlled by means of the width control device 94.

The rearward movement of the shoulder joint 34 (FIG. 3d) urges rotation of the clavicle 30 in the horizontal plane about the acromioclavicular joint 42, such that the first, or medial end 35 of the clavicle 30 is urged anteriorly. This movement of the clavicle 30 opens the gap between it and the first rib 26a, thereby reducing or possibly eliminating any compression taking place of the neurovascular structures 44 (FIG. 1f) that pass therethrough.

Additionally, the movement of the clavicles 30 moves the manubrium sternum forward (anteriorly) increasing the distance, or ‘diameter’ between the manubrium sternum and the anterior border of the body of T3 (the third thoracic vertebra), thereby alleviating (at least in some individuals) compression of certain veins, such as the brachiocephalic vein.

Additionally, the rearward movement of the shoulder joint 34 and any medial movement of the shoulder joint 34 adducts the scapula 22 (moves the scapula 22 medially). The movement of the scapula 22 medially, rotates the clavicle 30 about its own axis in a direction that is clockwise from a viewpoint facing the side of the right shoulder of the wearer 10 and counterclockwise from a viewpoint facing the side of the right shoulder of the wearer 10.

Additionally, the resulting medial movement of the scapula 22 elevates the pectoralis minor tendon 47 (FIG. 1d), reducing any compression that may take place on the neurovascular structures 27 (FIG. 1d) that pass underneath.

Additionally, the rearward movement of the shoulders 15 urges the spine 24 of the wearer 10 towards its neutral position, shown in FIG. 1a. As a result, pressure is redistributed between the vertebrae and the discs of the spine 24 in such a way as to reduce any bulging of the discs.

It is optionally possible for the rear portions 50b (FIG. 3c) of the straps 50 or for the anchor member 48 to extend along a path that takes them over the scapulae 22 (FIG. 3d) so that the tensile force that they are adapted to carry causes them to compress the scapulae 22 into the back to inhibit the non-neurogenic winging that can occur and to help correct scapular ptosis.

At least one of the ends 76 and 78 of the straps 50 may be removably connected to the anchor member 48 to facilitate the donning and removal of the support device 46. For example, as shown in FIG. 3a, the second ends 78 of the straps 50 may connect removably to the front 54 of the anchor member 48 by means of a connector 80. The connector 80 may be made up of any suitable structure. For example, the connector 80 may include a hook-and-eye portion 81 and a hook-and-loop portion 82. The hook-and-eye portion 81 includes a set of hooks 81a, (which may include as few as one hook 81a), which may, for example, be positioned on each of the straps 50, and sets of eyes 81b (whereby each set may include as few as one eye 81b), which may be positioned on the anchor member 48 for receiving the hooks 81a on the straps 50. The hook-and-loop portion 82 is positioned proximate each hook-and-eye portion 81 to prevent the hook-and-eye portion 81 from separating in the event that one or both of the straps 50 goes slack momentarily. The hook portions, shown at 82a may be positioned on the straps 50, for example, and the loop portions, shown at 82b may be positioned on the anchor member 48.

Preferably, the effective length of the front portions 50a of the straps 50 can be adjusted. For example, the ends 76 and 78 may be connectable to the anchor member 48 at a plurality of positions, thereby permitting the effective length of the straps 50 to be adjusted. For example, a plurality of sets of eyes 81b may be provided at different distances from the upper edge 55a of the anchor member 48 for receiving the hooks 81a from each of the straps 50. Alternatively, or additionally, a plurality of sets of hooks 81a may be provided at different distances from the second ends 78 of the straps 50. Thus, the effective length of the straps 50 can be adjusted by changing which set of eyes 81b receive the hooks 81a. The hook-and-loop connector portion 82 is connectable regardless of which set of eyes 81b are selected for receiving the hooks 81a.

Alternatively, the effective length of the straps 50 may be made adjustable by any other suitable means. For example, the straps 50 may include a length adjustment buckle, similar to that found on the shoulder straps of backpacks, or similar to that found on a belt.

As a result of the tension carried in the straps 50, some downward force will be exerted on the shoulders 15. The straps 50 avoid exerting any downward force on the clavicles 30 in order to avoid compression of the neurovascular structures 44 in the gap.

The shoulder-engaging portion 86 of each strap 50 has a medial edge, which is shown at 87, and which represents the medial limit to any downward force exerted by each shoulder-engaging portion 86 on the clavicle 30. It can be seen in FIG. 3d that the medial edge 87 of the shoulder-engaging portion 86 is positioned laterally of the medial edge 88 of the acromioclavicular joint 42, so that no downward force is applied to the clavicle 30. It is optionally possible for the medial edge 87 of the shoulder-engaging portion 86 to be positioned as far medially as the medial edge 88 of the acromioclavicular joint 42. Put another way, the shoulder-engaging portions 86 engage the shoulders 15 of the wearer 10 no further medially than the medial edges 88 of the acromioclavicular joint 42 of the wearer 10.

For the purposes of clarity, the medial edge 87 referred to above relates to the medial edge of the shoulder-engaging portion 86 only where it is relevant to the potential for compression of the clavicle 30. Therefore, it is not important whether portions of the shoulder-engaging portion 86 that are anterior or posterior to the clavicle 30 lie medially to the acromioclavicular joint 42, since they would not compress the clavicle 30 during use under normal conditions.

By contrast, certain posture support garments of the prior art engage the shoulders 15 medially of the acromioclavicular joints 42 and as a result they exert a downward force on the clavicles 30, which among other things, can result in compression of the underlying neurovascular structures 44 whether or not the shoulders 15 are moved rearward. Additionally, compression of the scalene muscles and the trapezius muscles can occur with some prior art posture support garments, which can cause discomfort or pain for the wearer.

The shoulder-engaging portions 86 may have a generally cupped shape in a front or rear elevation view, for cupping the shoulder joints 34, thereby inhibiting the straps 50 from moving medially on the wearer 10, and from slipping downwards along the arms 14 of the wearer 10. The cupped shape may be formed any suitable way. For example, the cupped shape may be formed by an arm-parallel portion 89 and a shoulder-parallel portion 90, which meet along a seam 92. Alternatively, the cupped shape may be formed in any suitable way.

In the embodiment shown in FIG. 3a, the straps 50 are integral at their first ends 76 with the anchor member 48. It is alternatively possible, however, for the straps 50 to be separate pieces which are joined removably or fixedly to the anchor member 48.

The path of each of the straps 50, (ie. passing between the front 54 of the anchor member 48 and the rear 56 of the anchor member 48 over one of the shoulders 15), is such that the straps 50 avoid passing across the anterior axillary fold 95. By avoiding passing across the fold 95, the straps 50 (and the entire support device 46) avoid compression of the lateral edge of the fold 95.

In addition to the above, it will be noted that the posture support device 46 does not include any force-carrying members that encircle the thorax either alone or in combination, thereby facilitating expansion of the rib cage that takes place during breathing by the wearer 10.

Scapular pads may be positioned in the back 56 of the anchor member 48 to enhance correction of scapular ptosis. The scapular pads may be triangular and increase the pressure on the shoulder blade moving them tighter to the back of the chest wall and in toward the midline of the thoracic spine. In one embodiment, they may be arranged such that the base of the triangle is vertical and the apex points toward the spine 24. The base of the triangle may be in the plane of the junction of the back side of the arm 14 and the axilla 93. Depending on the comfort introduced by the compression, thicker or thinner pads can be used. The pads may be made of a variety of materials.

An optional guide strap 105 (FIG. 3e) may be included to assist in donning the support device 46. The guide strap 105 may be removable.

It is optionally possible to include one or more strap fixing members 103 for inhibiting migration of the straps 50 on the wearer 10 during use. For example, the optional guide strap 105 shown in FIG. 3e may act as a strap-to-strap connector and may thus constitute a strap fixing member 103. The strap-to-strap connector 105 connects between the first and second straps 50 proximate where they engage the shoulders 15 of the wearer 10, and thereby inhibits the straps 50 from falling off the shoulders 15 of the wearer 10. The strap-to-strap connector 105 may have any suitable configuration for that purpose. For example, the strap-to-strap connector 105 may comprise a panel of material that extends across the chest of the user between the two straps 50. During use of the support device 46, the strap-to-strap connector 105 would provide some resistance to the spreading apart of the straps 50, which would therefore inhibit the straps 50 from falling off the shoulders 15 of the wearer 10.

The strap-to-strap connector 105 is preferably made from a material that has relatively little elasticity, so that it does not apply a force urging the straps 50 towards each other when the device 46 is being worn, so as not to encourage the straps 50 to migrate up towards the neck of the wearer 10. In order to fit wearers 10 having different shoulder widths, the strap-to-strap connector 105, if present could be made to be adjustable in length by some suitable means.

The strap-to-strap connector 105 may substantially cover the entirety of the space between the first and second straps 50 on the front of the support device 46. In other words, the strap-to-strap connector 105 may extend over the space defined by the straps 50, the anchor member 48 and the planned neckline for the wearer 10.

It is optionally possible for the strap-to-strap connector 105 to extend between each strap 50 and some point on the anchor member 48.

As shown in FIG. 3f, it is optionally possible for the strap-to-strap connector 105 to be present on the back portion of the straps 50 (ie. along the back 18 of the torso 12 of the wearer 10), instead of being present on the front 16 of the torso 12. It is a further alternative for the support device 46 to include two strap-to-strap connectors 105, one on the back 18 and one on the front 16.

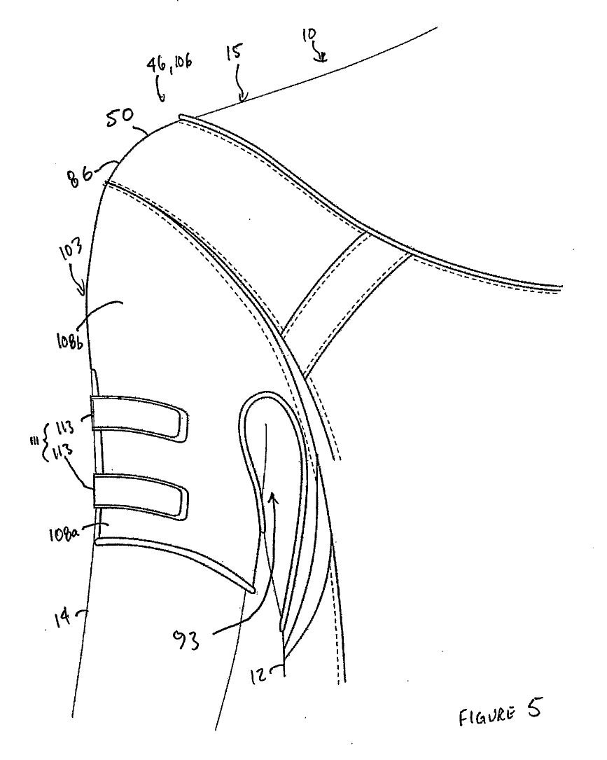

Referring to FIG. 5, an alternative strap fixing member 103 is shown. The strap fixing member 103 may be a sleeve portion 108 which is worn on each arm 14 of the wearer 10. The sleeve portion 108 connects to the shoulder-engaging portion 86 and grips the arm 14 sufficiently to retain the shoulder-engaging portion 86 in place substantially regardless of the position of the arm 14 or torso 10 of the wearer 10. As a result, when the wearer 10 moves in a way that urges the shoulder-engaging portion 86 out of position, the sleeve portion 108 substantially prevents it from moving. Any other retaining member could alternatively be used in place of the sleeve portions 108. The sleeve portions 108 need only extend some relatively small fraction of the length of the arm 14 of the wearer 10.

Each sleeve portion 108 includes a wrap-around portion 108a that wraps around an arm 14 of the wearer 10, however, that portion 108a is positioned below the axilla 93 to avoid compression of the anterior axillary fold 95 (FIG. 1g) and compression of the sensitive portions of the axilla 93 generally. The wrap-around portions 108a are connected to the shoulder-engaging portions 86 of the straps 50 (only strap 50 is shown in FIG. 5) by connector portions 108b, which do not wrap around the arms 14 of the wearer 10, and which may, for example, extend along the laterally outside surfaces of the arms 14.

The sleeve wrap-around portion 108a may be provided with a sleeve circumference adjustment device 111 which permits the effective circumference of the sleeve wrap-around portion 108a to be adjusted so that it has sufficient grip on the arm 14 to permit it to retain the shoulder-engaging portions 86 in place, without causing undue discomfort, or pain to the arm 14 and without causing undue compression of any neurovascular structures in the arm 14, particularly those structures on the upper portion of the arm 14 facing the side 27 of the thorax 21, where the arm 14 is relatively sensitive. The sleeve circumference adjustment device 111 may have any suitable structure. For example, it may include one or more hook and loop connectors 113 on the sleeve wrap-around portion 108a. Alternatively, it may include one or more buckles and adjustment straps on each sleeve wrap-around portion 108a.

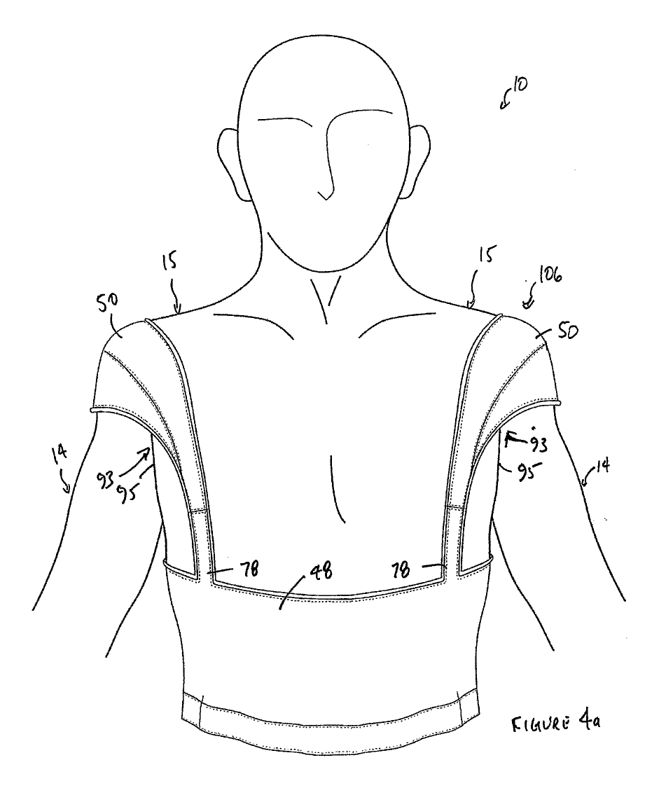

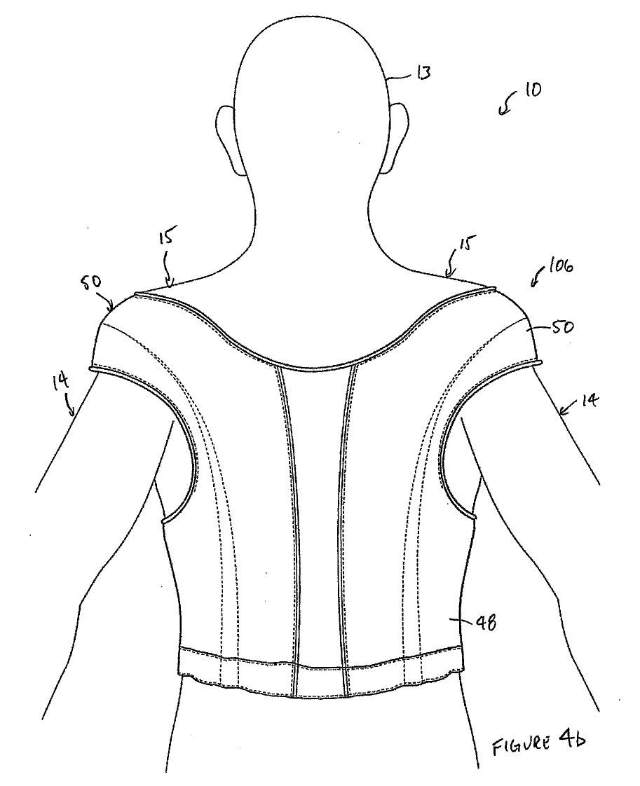

Reference is made to FIGS. 4a and 4b, which show a posture support device 106 in accordance with an alternative embodiment of the present invention. The posture support device 106 may be similar to the posture support device 46 (FIG. 3a), except that the posture support device 106 may lack the connectors 60 and 80 shown in FIGS. 3a, 3b and 3c, and may instead have permanent connections in their place. For example, the anchor member 48 may be unopenable, and may instead be present as a continuous loop. Similarly, the ends 76 (FIG. 4a) and 78 (FIG. 4b) of the straps 50 may all be permanently joined to the anchor member 48. The support device 106 may optionally include or omit the width control device 94 (FIG. 3a).

It is also optionally possible for the support device 106 to include the connector 60 (FIG. 3a) while omitting the connectors 80 (FIG. 3a), or alternatively to include the connectors 80 (FIG. 3a) while omitting the connector 60 (FIG. 3a).

In order to be able to be put on the posture support device 106, the material from which it is made could be selected to have sufficient elasticity to permit the anchor member 48 to be pulled over the head 13 and shoulders 15 of the wearer 10 to its target position on the torso 12 of the wearer 10. Alternatively the device 106 could be made to have sufficient elasticity (through construction from a suitable material) to permit the wearer 10 to step into it and pull it upwards into position.

The optional guide strap 105 and the optional strap fixing member or members 103 (FIGS. 3e and 3f, which may be made up of the strap-to-strap connector 105 (ie. the guide strap 105) (FIGS. 3e and 3f, may be incorporated into the support device 106.

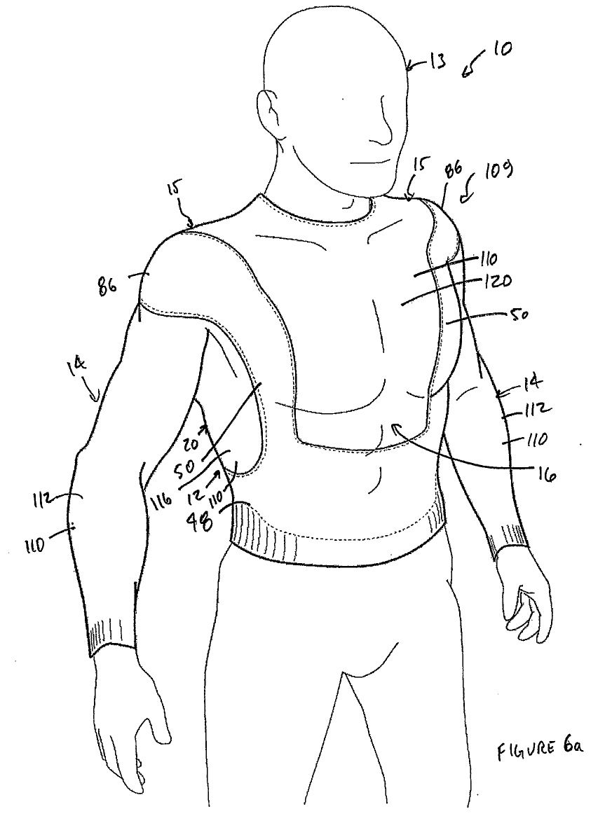

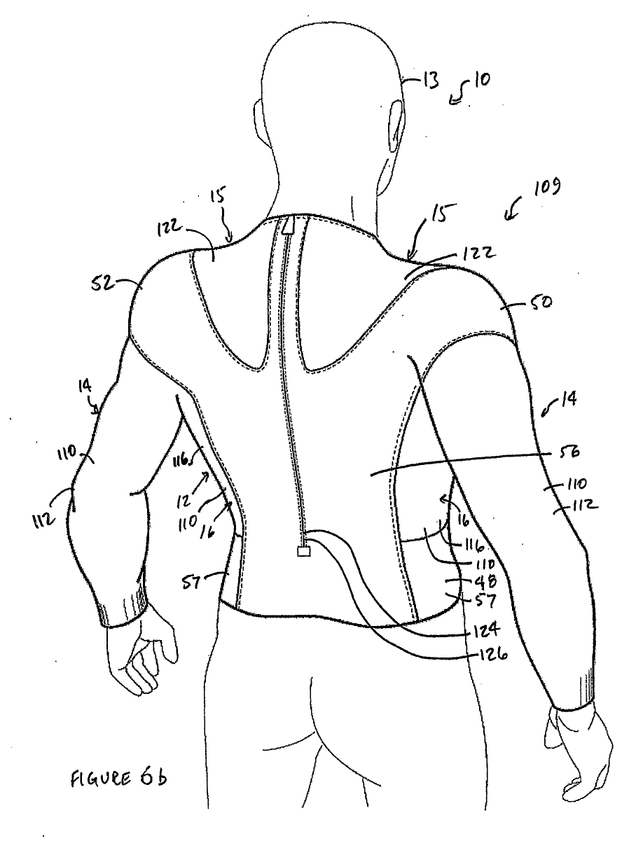

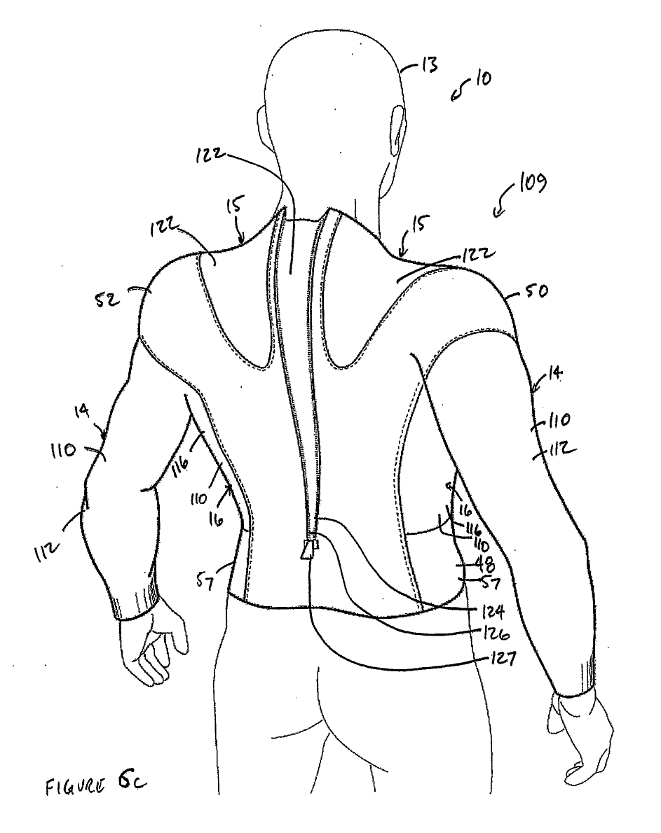

Reference is made to FIGS. 6a and 6b, which show a posture support device 109 in accordance with an alternative embodiment of the present invention. The posture support device 109 may be similar to the posture support devices 46 (FIGS. 3a, 3b and 3c) or 106 (FIGS. 4a and 4b), except that the posture support device 109 is structured so that it may be worn as a top instead of as an undergarment.

The posture support device 109 includes the anchor member 48, the first and second straps 50, and further includes a plurality of covering panels 110. The covering panels 110 are connected to the anchor member 48 and the first and second straps 50 and extend across spaces therebetween to cover portions of the body of the wearer 10 that are not covered by the anchor member 48 or the first and second straps 50.

The covering panels 110 connect to the anchor member 48 and straps 50 in such a way so that they exert substantially no force related to postural support on the wearer 10. In other words, the covering panels 110 cover the body of the wearer 10 without acting as force transfer members, as opposed to the anchor member 48 and the first and second straps 50 which do act as force transfer members and which do exert forces related to postural support on the wearer 10. Preferably, the covering panels 110 exert substantially no forces at all on the wearer 10, however, it is possible that they will exert some force on the wearer 10 in certain circumstances. For example, when the wearer 10 positions his or her arms 14 or body in certain positions, it is possible that the covering panels 110 may exert a force on the body of the wearer 10, however, such a force is not a force associated with posture support. As another example, it is possible that the covering panels will be made to fit relatively snugly against the skin of the wearer 10, however they may be made from a relatively elastic material that stretches easily. As a result of their snug fit and their elasticity, they may exert a small force on the body of the wearer 10 at all times while the support device 109 is worn, however this force is relatively small and is not related to postural support.

The covering panels 110 may be configured in any suitable way to cover the body of the wearer 10. For example, the covering panels 110 may include right and left sleeves 112, right and left side panels 116 (the right side panel 116 is shown in FIG. 6a and the left side panel 116 is shown in FIG. 6b), and a front panel 120 (FIG. 6a). Any of the panels 112, 116, and 120 may be made up of one or more pieces of material as necessary. The material of the panels 112, 116, and 120 may be selected to have any desired characteristics, such as, for example, breathability, softness or moisture absorption.

Each side panel 116 extends along a side 16 of the torso 12 of the wearer 10, connecting between the front portion 50a of a strap 50, the rear portion 50b of the same strap 50 and a side 57 of the anchor portion 48. Each sleeve 112 connects between one of the side panels 116, and also connects to the shoulder-engaging portion 86 of the first strap 50. It is optionally possible for the side panels 116 to connect along the entirely of the outside edges of the first and second straps 50 and for the sleeves 112 to attach entirely to the side panels 116 instead of attaching partially to the first and second straps 50.

The front panel 120 attaches between the first and second straps 50 and attaches at its bottom to the front 54 of the anchor member 48. The front panel 120 extends up to the shoulders 15 of the wearer 10 and forms the front of the neck aperture of the posture support device 109. Depending on the extent of coverage of the back 56 of the anchor portion 48 over the back 18 of the torso 12 of the wearer 10, one or more back panels 122 may be provided which may, for example, connect between the back 56 of the anchor portion 48, the first and second straps 50 and the front panel 120. The back panels 122 may define at least a portion of the back portion of the neck aperture of the support device 109.

The front and back panels 120 and 122 each connect to both straps 50 proximate the shoulders 15. As a result, the front and back panels 120 and 122 each act as strap fixing members.

As shown in FIG. 6b, the back 56 of the anchor portion 48 includes a width control device 124, which may, for example, be a zipper 126. The zipper 126 may extend to the very top of the back of the support device 109. A back panel 122 may be positioned between the edges of the zipper 126, which can protect the skin of the wearer 10 during closure of the zipper 126.

The zipper 126 include a handle 127, which may be provided with a sufficiently long pull-cord to extend over the shoulders to the front of the wearer 10, thereby permitting the wearer 10 to close the zipper 126 by themselves while wearing the support device 109.

As an alternative to a zipper, it is possible for the support device 109 to incorporate a width control device 124 that is similar to that shown for the support device 46 shown in FIG. 3b, which incorporates a lace member and a set of apertures on either side of a separation.

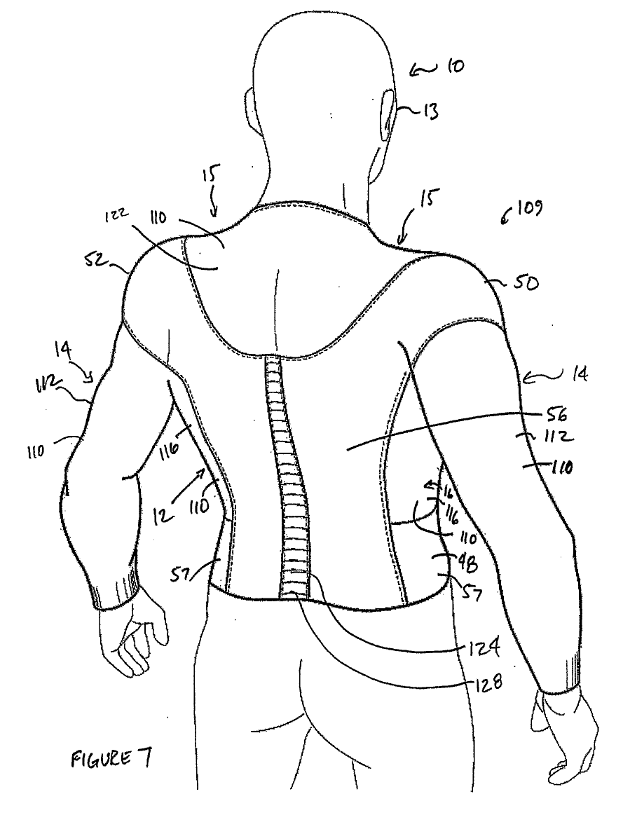

Reference is made to FIG. 7. Instead of a zipper, the width control device 124 could alternatively be a set of elastic members 128. The elastic members 128 stretch to permit the user to put on the support device 109, and contract towards their at rest lengths once the support device 109 is on the wearer 10, thereby urging the shoulders 15 rearward. An advantage of the elastic members 128 is that a second person is not required to assist the wearer 10, as could be necessary for embodiments including a zipper such as that shown in FIG. 5b, or embodiments including laces, such as that shown in FIG. 3b.

It is optionally possible that the front panel 120 would be replaced by the strap-to-strap connector 105. Alternatively, it is optionally possible for the support device 109 to include a strap-to-strap connector 105 that does not cover the entirety of the space between the first and second straps 50, and to further include a front panel 120, to cover the portion or portions of the front of the torso 12 not covered by the strap-to-strap connector 105.

Depending on the purpose of the support device 109, the device 109 may be configured to substantially conceal the fact that it is a posture support device so that it presents to others as simply being a piece of clothing, optionally fashionable clothing. Alternatively, the support device 109 may be configured for use as a sport garment, in which case, it may be more acceptable for it to be visually apparent that the support device 109 is acting as a posture support device and is not simply to cover the body of the wearer 10. Thus, particularly in the case where it is a sport garment, the support device 109 may include openable connectors 60 and/or 80 to facilitate donning and removal.

It is optionally possible for the support device 109 to have as few as one covering panel 110, which covers a single portion of the body of the wearer 10.

In any of the embodiments disclosed herein, it is optionally possible to provide strengthening members, such as, for example, vertically extending semi-rigid bars, at selected positions in the support device. These strengthening members facilitate the transmission of forces throughout some or all of the support device.

For all of the support devices provided above, it is contemplated that several sizes of the device can be provided so that the device can be used by a relatively wider portion of the general population.

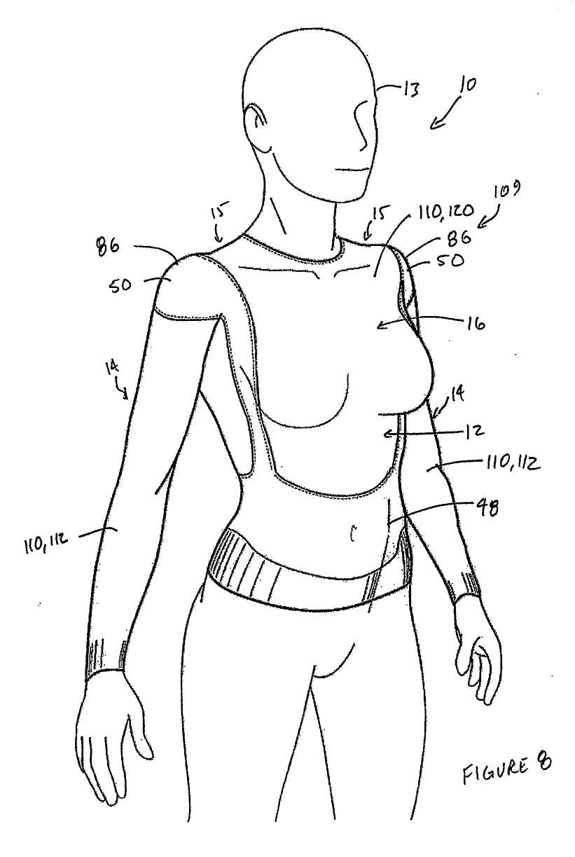

The posture support device 46, 106 or 109 can be worn by a male, as shown in FIGS. 3b, 4a and 6a. It is alternatively possible for the posture support device 46, 106, 109 to be worn by a female wearer 10, shown in FIG. 8 (by way of example, the posture support device 109 is shown in that figure). In such a case, it may be preferable for the first and second straps 50 to extend along the front 16 of the torso 12 sufficiently laterally so as to avoid extending over the front of the breasts of the wearer 10.

The benefits provided by the posture support devices 46, 106 and 109 shown and described herein may not exist for every individual. However, some individuals will receive at least some of the benefits of the support devices 46, 106 or 109.

The posture support device 46, 106, 109 can be worn 24 hours a day, if desired. For example, with loose clothing, the front circumference adjustment device 67 and the sleeve circumference adjustment devices 111 can be adjusted for comfort or for increased postural stability in situations that create prolonged postural strain such as typing, driving, and in some cases sleeping.

In another aspect of the present invention, a method is provided for selecting, fitting, and ordering a body posture device. The order may be achieved through a variety of methods including but not limited to using a web interface, a telephone, or the like. In one embodiment, the steps may comprise:

1. Determine method of payment once order has appeared in the chartware or other means of notice.

2. In order to obtain authorization from comp: a) use the “click-on” vignettes to illustrate the device and describe its use and b) click on a statement that indicates the specific need that the selected patient has for the device. Fax this with copy of order to the carrier in conjunction with phone request.

3. If Medicare, then use the Medicare billing numbers and submit.

4. If private, fax the order but secure payment before issuing the device.

The ordering process would in one embodiment comprise:

1. Copy “brace measurements” form in documents and click on any explanatory statements that are needed to anticipate or answer the carrier's questions. These will be in a file within the Brace Measurements folder but also may probably be in chartware as a set of snippets justifying the need for the device.

2. Once authorization has been obtained from comp, then fax the order form with measurements including clicking to add any or all of the added features: night yoke, scapular pads, or interscapular bolsters, and if so, which size.

3. Record the required tracking information on the master order list that indicates payer, date of authorization, date order faxed, any tracking number if we agree to use one and later indicate once the completed item comes to us for stock indicating whether it has the appropriate attachments or not.

While the above description constitutes the preferred embodiments, it will be appreciated that the present invention is susceptible to modification and change without departing from the fair meaning of the accompanying claims.

1. The posture support device as claimed in claim 20
wherein each strap has a first end and a second end and connects to the anchor member at the first and second ends.

2. A posture support device as claimed in claim 1, wherein each shoulder-engaging portion has a front and a rear, and each strap extends downward from the front of the shoulder engaging-portion to the second end, wherein the anchor member has a front for engaging the front of the wearer and wherein the second end of each strap is connected to the front of the anchor member.

3. A posture support device as claimed in claim 1, wherein each shoulder engaging portion has a front and a rear, and each strap extends downward at a non-vertical angle from the rear of the shoulder engaging-portion to the first end, so that, in use, the tensile force that the straps are adapted to carry causes the straps to urge the shoulder joints medially.

4. A support device as claimed in claim 1, wherein the path for each strap is configured to extend across one of the scapulae of the wearer, and wherein, in use, the tensile forces which the straps are adapted to exert forces on the medial edges of the scapulae urging the medial edges into the back of the wearer.

5. A support device as claimed in claim 1, wherein the first and second straps are removably connectable to the anchor member.

6. A support device as claimed in claim 5, wherein the first and second straps each have an effective length that is adjustable.

7. A support device as claimed in claim 6, wherein at least one of the ends of the first and second straps is connectable at a plurality of positions to the anchor member.

8. A support device as claimed in claim 1, wherein the posture support device has a shoulder-width associated therewith, and wherein the anchor member has a back and the back of the anchor member has a shoulder width adjustment device for adjusting the shoulder width of the posture support member to accommodate wearers having a range of shoulder widths.

9. A support device as claimed in claim 8, wherein the back of the anchor member includes a left back portion and a right back portion and wherein a generally vertically extending separation extends downwards from the top of the back of the anchor member and separates the left and right back portions along at least a portion of the height of the back of the anchor member, and wherein the left and right back portions each include a column of tie apertures proximate the separation, and wherein the shoulder width adjustment device includes at least one tie member that passes through the tie apertures.

10. A support device as claimed in claim 1, wherein the anchor member has an effective circumference and has a circumference adjustment device for adjusting the effective circumference.

11. A support device as claimed in claim 1, wherein the first and second shoulder-engaging portions are cupped to engage the shoulder joints of the wearer.

12. A support device as claimed in claim 1, further comprising at least one covering panel, wherein the at least one covering panel is connected to at least one of the anchor member, the first strap and the second strap while transferring substantially no force between the wearer and any of the anchor member, the first strap or the second strap, wherein the at least one covering panel is configured to cover portions of the body of the wearer which the anchor member and the first and second straps are not configured to cover.

13. A support device as claimed in claim 1, further comprising a sleeve portion connected to each shoulder-engaging portion, wherein each sleeve has a wrap-around portion that is configured to wrap around the arm of the wearer and grip the arm of the user to inhibit migration of the associated shoulder-engaging portion during use.

14. A support device as claimed in claim 1, and wherein the wrap-around portion is configured to be spaced from the anterior axillary folds of the wearer.

15. A posture support device for a wearer, comprising:
an anchor member, wherein the anchor member is fittable around the torso of the wearer;
a first shoulder engaging portion and a second shoulder engaging portion, wherein each of the shoulder engaging portions are configured to urge the shoulders of the wearer rearward while avoiding exerting a downward force on the clavicles of the wearer, and wherein each of the shoulder engaging portions are configured to engage the shoulders of the wearer no further medially than the acromioclavicular joints; and,
wherein the posture support device avoids crossing over the anterior axillary folds of the user.

16. The posture support device of claim 15, further comprising covering panels connecting connected to the anchor member.

17. The posture support device of claim 16, wherein the covering panels are made from an elastic material.

18. The posture support device of claim 16, wherein the covering panels comprise a right sleeve, a left sleeve, a right side panel a left side panel and a front panel.

19. The posture support device of claim 15, further comprising vertically extending semi-rigid bars.

20. The posture support device of claim 15, further comprising a first strap and a second strap wherein the first strap comprises the first shoulder-engaging portion, and the second strap comprises the second shoulder-engaging portion.
